(19) United States
(12) Patent Application Publication
(10) Pub. No.: US 2026/0083959 A1
(43) Pub. Date: Mar. 26, 2026

(54) DEVICE AND METHOD FOR MONITORING THE STATE OF HEALTH OF A PATIENT

(71) Applicant: KARDION GMBH, Stuttgart (DE)

(72) Inventors: Hardy Baumbach, Stuttgart (DE); Julian Kassel, Böblingen (DE); Tjalf Pirk, Stuttgart (DE); Inga Schellenberg, Stuttgart (DE); Martina Budde, Karlsruhe (DE); Thomas Alexander Schlebusch, Renningen (DE)

(21) Appl. No.: 19/404,825

(22) Filed: Dec. 1, 2025

Related U.S. Application Data
(63) Continuation of application No. 17/266,056, now Pat. No. 12,508,418.

(30) Foreign Application Priority Data
Aug. 8, 2018 (DE) 10 2018 213 350.6

(51) Int. Cl. A61M 60/585; A61M 60/139; A61M 60/17; A61M 60/178; A61M 60/216; A61M 60/422; A61M 60/531; A61M 60/592

(52) U.S. Cl. CPC A61M 60/585; A61M 60/139; A61M 60/17; A61M 60/178; A61M 60/216; A61M 60/422; A61M 60/531; A61M 60/592

(57) ABSTRACT

The disclosure relates to a device (150) for monitoring the state of health of a patient (100), wherein the device (150) comprises an input interface (160) for inputting a first pressure signal (145) and a second pressure signal (155) and a processing unit (165) for processing the first pressure signal (145) and the second pressure signal (155) in order to determine a processing value (170) in order to monitor the state of health of the patient (100) based the processing value (170).

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application is a continuation of U.S. Ser. No. 17/266056, filed on Oct. 13, 2021, titled DEVICE AND METHOD FOR MONITORING THE STATE OF HEALTH OF A PATIENT, which is a National Stage Entry of PCT/EP2019/071245, filed on Aug. 7, 2019, titled DEVICE AND METHOD FOR MONITORING THE STATE OF HEALTH OF A PATIENT, which claims priority to German Patent Application No. 10 2018 213 350.6, filed on Aug. 8, 2018, the entire contents of which are incorporated by reference herein for all purposes and forms a part of this specification.

### BACKGROUND

### Field

The disclosure is based on a device or a method of a type of the independent claims. The present disclosure also relates to a computer program.

### Description of the Related Art

In patient support systems, such as cardiac support systems, for example, a diagnosis of the patient's state of health is made on the basis of numerous measured values. The measurement results are often not precise and stable enough, however, and therefore do not allow a reliable and predictive assessment of the patient's state of health to the extent necessary or possible.

Based on this, the underlying object of the disclosure is to further improve the devices and methods known in the state of the art and to provide the most precise possible acquisition of relevant parameters for patient monitoring and for operating a cardiac support system.

### SUMMARY

With this in mind, the approach presented here introduces a device, a method, and a corresponding computer program according to the independent claims. Advantageous further developments and improvements are possible using the measures listed in the dependent claims.

The approach presented here provides a device for monitoring the state of health of the patient, wherein the device has the following features:
an input interface for inputting a first pressure signal and a second pressure signal; and
a processing unit for processing the first pressure signal and the second pressure signal to determine a processing value in order to monitor the state of health of the patient based on said processing value.

A state of health of a patient can be understood to be a parameter or an indication that describes or depicts a functionality of an organ of a patient, for example, and thereby possibly provides an indication of physical impairment now or in the future which may also require treatment by a physician. In the present case, a pressure signal can be understood to be a value that represents a pressure in a medium. For example, the pressure signal can correspond to a blood pressure or an air pressure outside a patient. In the present case, a processing value can be understood to be a value or parameter that depicts or represents an indication or a degree of a state of health of the patient. This processing value makes easy, stable, reliable and long-term monitoring of the patient's state of health possible.

The approach presented here is based on the knowledge that a patient's state of health can be determined very reliably and easily by processing two pressure signals, for example in the form of a comparison or a calculation of the difference. For example, two pressure signals can be related to one another in terms of time or absolutely, for example to determine a pressure difference or a parameter of a pressure wave, for example of blood in a vessel of the patient, in order to thereby obtain an indication of an elasticity of a vascular wall or a degree of functionality of an organ of the patient and thus be able to monitor the state of health of the patient. The patient's state of health can thus be monitored flexibly and cost-effectively from at least one aspect.

One embodiment of the approach proposed here advantageously includes the processing unit being configured to process the first pressure signal as a blood pressure value in a ventricle of the heart and the second pressure signal as a blood pressure value in the aorta of the patient. A blood pressure difference and/or a pulse wave velocity of a blood pulse wave or an elasticity of at least one blood vessel can be determined as the processing value to monitor the state of health of the patient. Such an embodiment of the approach proposed here has the advantage that a parameter enabling a statement to be made about the patient's state of health with a high degree of precision can be obtained as a processing value by technically simple and inexpensive means.

Also beneficial is an embodiment of the approach proposed here, in which the input interface is further configured to input a cardiac support blood flow value of an activated cardiac support system which represents a blood flow induced by the cardiac support system. The processing unit can furthermore also be configured to use the cardiac support blood flow value as the processing value to determine a performance value of the heart which corresponds to a pump power of the patient's heart minus the cardiac support blood flow value. Such an embodiment of the approach proposed here has the advantage of determining a residual power of the heart as the organ of the patient to be supported when an activated cardiac support system is being used, and from that being able to infer the current performance of the heart and predict any medical measures that may be required.

According to another embodiment of the approach proposed here, the input interface and the processing unit can also be configured to be disposed and operated outside the body to determine the processing value. Such an embodiment of the approach proposed here has the advantage that an energy supply of the input interface and/or the processing unit can be designed in a technically very simple manner, so that there is no need to route an energy supply line to a location in the patient that may be problematic to reach. The space required for a unit installed in the patient can furthermore be reduced if the pressure signals are evaluated outside the patient.

According to a further embodiment of the approach proposed here, the input interface and/or the processing unit can be designed as units that can be disposed or are disposed outside a building in which the patient is located, in particular wherein the input interface and/or the processing unit are designed as units of a cloud server or a computer unit that can be contacted via an Internet connection. Such an embodiment of the approach proposed here has the advantage of central monitoring of the state of health of the patient, whereby algorithms for linking the pressure signals to determine the processing value can also be changed very quickly and easily, for example, if this appears to be necessary based on more recent medical or physiological findings. At the same time, the patient can be monitored quickly and in real time and, if it is detected that the patient is in a critical state of health, for example, emergency services can be alerted.

In order to be able to undertake a longer-term monitoring of the patient or the patient's state of health, according to a further embodiment of the approach proposed here, the input interface and/or the processing unit can be configured to input a first and a second pressure signal at repeated time intervals and determine a processing value, wherein the processing unit is further configured to store the determined processing values and compare them with one another, and in particular output an alarm signal if one or more of the processing values exceeds a threshold value.

To obtain an absolute value of the blood pressure in the patient, for example, according to a further embodiment of the approach presented here, the input interface can further be configured to input an ambient air pressure value as the first pressure signal, which in particular represents an ambient air pressure in the immediate vicinity of the patient, wherein the processing unit is configured to determine a blood pressure value of the patient relative to the ambient air pressure as the processing signal. The ambient air pressure can be subtracted from the blood pressure sensor value to determine the processing signal, for example. Such an embodiment of the approach proposed here is advantageous, for example, when the ambient air pressure around the patient changes. This can be caused by the effect of an air conditioning system, a pressure chamber, a change in weather conditions and/or geographic altitude, etc., on the environment or surroundings of the patient, for example. In such an embodiment, it is then possible to detect a falsification of the measurement result via a change in the ambient air pressure and take this into account, for example, when assessing the state of health.

Also conceivable is an embodiment of the approach proposed here in which the processing unit is configured to output a control signal to a cardiac support system based on the processing value and/or to output a data transmission signal to a central processing unit and/or a cloud server via an Internet connection as a processing value, in particular wherein the data transmission signal comprises at least one piece of information which was obtained from the first pressure signal and/or the second pressure signal by means of a data compression method. Such an embodiment of the approach proposed here has the advantage of making it possible to react very quickly to a change in the patient's state of health and/or a strain situation, to thus be able to bring about an immediate improvement in the patient's living situation.

Another embodiment of the approach presented here proposes a method for monitoring a state of health of the patient, wherein the method comprises the following steps:
inputting a first pressure signal and a second pressure signal; and
processing the first pressure signal and the second pressure signal to determine a processing value in order to monitor the state of health of the patient based on said processing value.

This method can, for example, be implemented in software or hardware or in a mixed form of software and hardware in a control device, for example.

The approach presented here further creates a device that is configured to carry out, control and/or implement the steps of a variant of a method presented here in corresponding devices. This design variant of the disclosure in the form of a device also makes it possible to achieve the underlying object of the disclosure quickly and efficiently.

For this purpose, the device can comprise at least one computing unit for processing signals or data, at least one memory unit for storing signals or data, at least one interface with a sensor or an actuator for inputting sensor signals from the sensor or for outputting data or control signals to the actuator, and/or at least one communication interface for inputting or outputting data embedded in a communication protocol. The computing unit can be a signal processor, a microcontroller or the like, for example, whereas the memory unit can be a flash memory, an EEPROM or a magnetic memory unit. The communication interface can be configured to input or output data in a wireless and/or wired manner, whereby a communication interface that can input or output wired data can, for example, input or output said data electrically or optically from or to a corresponding data transmission line.

In the present case, a device can be understood to be an electrical device that processes sensor signals and outputs control and/or data signals as a function of said sensor signals. The device can comprise an interface that can be hardware and/or software-based. In the case of a hardware-based configuration, the interfaces can be part of a so-called system ASIC, for example, which contains the various functions of the device. However, it is also possible for the interfaces to be separate, integrated circuits or consist at least in part of discrete components. In the case of a software-based configuration, the interfaces can be software modules that are, for example, provided on a microcontroller alongside other software modules.

A computer program product or computer program having program code which can be stored on a machine-readable carrier or storage medium such as a semiconductor memory, a hard drive memory, or optical memory and is used to carry out, implement, and/or control the steps of the method according to one of the embodiments described above is also advantageous, in particular if the program product or program is executed on a computer or a device.

### BRIEF DESCRIPTION OF THE DRAWINGS

Design examples of the approach presented here are shown in the drawings and explained in more detail in the following description. The figures show:

FIG. 1 an illustration of a patient implanted with an example of a cardiac support system which cooperates with a design example of the device for monitoring the state of health of the patient presented here;

FIG. 2 a schematic illustration of a cardiac support system to be equipped with the pressure sensors shown in FIG. 1;

FIG. 3 a block diagram of an example of a compression method for streaming data, for example a blood pressure curve, to a cloud; and

FIG. 4 a flow diagram of a method according to one design example.

### DETAILED DESCRIPTION

In the following description of favorable design examples of the present disclosure, the same or similar reference signs are used for the elements shown in the various figures, which have a similar effect, whereby a repeated description of these elements is omitted.

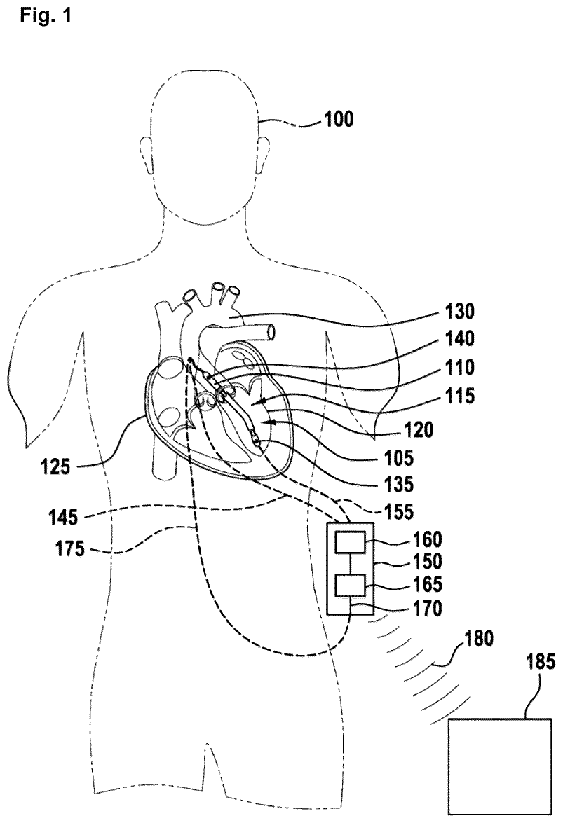

FIG. 1 shows an illustration of a patient 100 implanted with an example of a cardiac support system 105 which cooperates with a design example of the device for monitoring the state of health of the patient 100 presented here. The cardiac support system 105 is shown in FIG. 1 only as an example in order to describe corresponding components of said cardiac support system 105 in more detail. In addition to a blood pump driven by a motor 110, the cardiac support system 105 comprises a blood guiding element 115 which can bring blood from a ventricle 120 of the heart 125 into the aorta 130 of the patient 100. A first pressure sensor 135 and a second pressure sensor 140 are disposed in the blood guiding element 115 (or a part thereof or parts adjacent to it), for example, whereby the first pressure sensor 135 sends a first pressure signal 145 to a device 150 for monitoring a state of health of the patient 100 according to one design example of the approach proposed here. The second pressure sensor 140 sends a second pressure signal 155, for example, to the device 150. The first pressure sensor 131 and the second pressure sensor 140 are disposed at a predetermined distance from one another in the blood guiding element 115, for example, so that they can register the blood pressure, blood pressure fluctuations or a pulse wave of blood, for example. The first pressure sensor 135 can be configured to transmit the first pressure signal 145 to the device 150 in the form of electromagnetic waves, i.e., wirelessly. The second pressure sensor 140 can analogously be configured to also transmit the second pressure signal 155 to the device 150 wirelessly and/or in the form of electromagnetic waves. The device 150 comprises an input interface 160, by means of which the first pressure signal 145 and the second pressure signal 155 can be inputted. The inputted pressure signals 145 and 155 are forwarded to a processing unit 165, in which a processing value 170 is determined, on the basis of which a state of health of the patient 100 can then be monitored. Such a processing value 170 can, for example, be a transit time of the pulse wave of blood between the first pressure sensor 135 and the second pressure sensor 140. Alternatively or additionally, such a processing value 170 can also be a parameter representing an elasticity of vascular walls such as a wall of the aorta 130, so that, using said parameter or processing value 170, the state of health of the patient can then also be assessed with respect to the elasticity of vascular walls, for example, to identify deposits or calcifications on inner walls of the vasculature.

It is also possible, for example, for the device 150 to output a control signal 175 as a function of the processing value 170 to control the motor 110 of the cardiac support system 105 to be able to set a desired blood pressure, or a blood pressure appropriate for specific activities (e.g., climbing stairs), for the patient 100.

It is furthermore also conceivable for a data transmission signal 180 to be transmitted (for example via an Internet connection) to a central processing unit 185 (for example in the form of a cloud server) on the basis of the processing value 170 or the first data signal 145 and/or the second data signal 155, in order to enable or simplify notification of a physician or an evaluation of the pressure signals 145 or 155.

In the design example of the present disclosure shown in FIG. 1, the device 150 is depicted as a device that can be worn externally on the patient 100, for example attached to a belt of the patient 100. On the other hand, it is also conceivable for the device 150 to be designed as an integral component of the cardiac support system 105, so that the first pressure signal 145 and/or the second pressure signal 155 can be transmitted in a wired manner. In this case, however, it must be ensured that the energy supply of the components of the device 150 is secured, either by means of a long-life battery or a suitable rechargeable battery, by routing a respective energy supply line for the device 150 or by transmitting electrical energy via electromagnetic fields to the device 150, which is then implanted in the patient 100. In a further embodiment, the component 150 can also be subdivided, so that the input interface 160 is disposed in an implanted control device, while the processing unit 165 is placed on the belt outside the patient, for example coupled via a radio link.

As discussed in more detail in the following, one of the pressure sensors 135 or 140 can be disposed outside the patient 100 as well, for example in the device 150 as shown in FIG. 1, and register an absolute air pressure, by means of which a standardization of the pressure value obtained from the other pressure sensor(s), which then preferably represents a blood pressure, can be carried out. This makes it possible to very reliably ensure an absolute blood pressure value of the blood pressure of the patient 100, in which systematic errors caused, for example, by a change of the ambient air pressure of the patient 100 (for example, when changing floors in a high-rise building, weather-based air pressure changes or topographic elevation) have been compensated. The patient's state of health can thus be determined very reliably in different environmental scenarios.

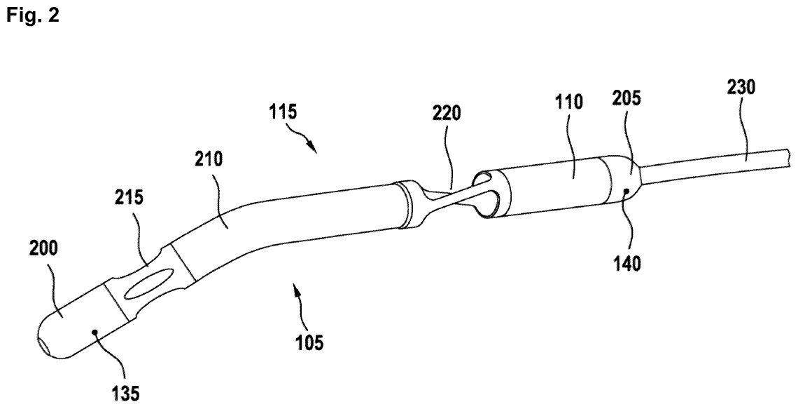

FIG. 2 shows a schematic illustration of a cardiac support system 105, which is to be equipped with the pressure sensors 135 and 140 shown in FIG. 1. The cardiac support system 105 can use absolute pressure sensors as the pressure sensors 135 and 140 to record a blood pressure curve including systole and diastole. One (or more) pressure sensor(s) 135 in a tip 200 of the cardiac support system 105 can record the pressure in the ventricle 120. If a further pressure sensor 140 is installed at the end 210 of the cardiac support system 105, both the aortic pressure and the differential pressure between the ventricle 120 and the aorta 130 can be determined. Since the measured values of the absolute pressure sensors as the pressure sensors 135 or 140 are a superposition of the blood pressure and the barometric ambient air pressure, according to one design example of the approach presented here, the ambient air pressure may be needed to determine the blood pressure as well. Said ambient air pressure can, for example, be provided by a separate system such as the device 150 shown in FIG. 1, but also by a smartphone, for example. The pressure signals can be processed in the control device or the device 150 as shown in FIG. 1, but also in cloud-based data processing if the device 150 is disposed in a corresponding cloud server 185 connected to the cardiac support system 105 via the Internet. To exclude tampering with data or control signals for the cardiac support system 105 as much as possible, the pressure signals 145 or 155 should be encrypted, for example using a cryptographic method, and sent to an extracorporeal evaluation unit such as the device 150 of FIG. 1. It is also conceivable for control signals to be sent from the extracorporeal evaluation unit, such as the device 150 of FIG. 1, to the cardiac support system 105 or components thereof, whereby here again these signals should advantageously be encrypted using a cryptographic method to protect against tampering. Continuous recording and evaluation of the blood pressure values allows an assertion to be made about the heart's 125 own capacity. The ability to determine the location of the patient 100 with a smartphone connected to the system or the device 150 provides the further advantages of a cloud-based evaluation of the blood pressure values.

FIG. 2 shows a schematic illustration of a cardiac support system 105. In the implanted state, the tip 200 and a suction hose 210 with an inlet region 215 for the blood are disposed in the ventricle. In the implanted state of the cardiac support system 105, all of the other parts beyond an outlet opening 220 are disposed in the aorta. The motor 110 provides blood circulatory support, whereby, in the illustration of FIG. 2, the motor 110 is disposed in the aorta. The “back end” 205 mentioned here and in the following is disposed the distal end of the motor 110. From there, an electrical lead 230 leads to a control unit or the device 150 according to one design example of the approach presented here. One or more absolute pressure sensors, such as the sensor 135, can be disposed in the tip 200 or another region of the system (200, 215, or 210) which, in the implanted state, is located in the ventricle. This makes it possible to determine the blood pressure in the ventricle.

One (or more) further pressure sensor(s) 140, which is/are disposed in the aorta, for example, can be used to measure the arterial blood pressure there. In principle, every part of the system in the aorta should be a possible application site for the pressure sensor(s) 140. According to another design example, the further pressure sensor 140 could also be integrated into the back end 205 of the motor 110. By using a plurality of pressure sensors 135, 140 which are disposed spatially apart from one another and, for example, at least partially in the region of the aorta (for example along a driveline), the propagation speed of pressure changes/pulse waves can be observed. If pressure sensors 135, 140 are used in both the ventricle and the aorta, an internal and/or an external control unit, for example the device 150 mentioned with reference to FIG. 1, can determine the differential pressure. Said differential pressure can be used, among other things, to set the motor output 110 and/or to determine the state of health of the patient 100.

Thus, using at least two pressure signals, the approach presented here can be used in several possible ways to examine the state of health of the patient 100 based on a processing value 170 determined from the pressure values or pressure signals 145 or 155. For example, the systolic and diastolic blood pressure can be determined in the ventricle 120 and in the aorta 130. A determination of the differential pressure between the ventricle 120 and the aorta 130 and/or an assessment of the heart's 125 own capacity/activity is conceivable as well. The determined processing value 170 can also be used to carry out an assessment of the elasticity of the blood vessels in the vicinity of the cardiac support system 105. It is also conceivable for a processing value 170 to be determined which enables an assessment of the pulse wave velocity as a measure for the condition of the patient 100 or an evaluation and calibration of the pressure signals of the pressure signals 145 and/or 155 in an extracorporeal control device. It is also advantageous if long-term recording and evaluation of blood pressure values takes place in a cloud or central learning for patient monitoring and possible early diagnosis of any changes in the state of health of the patient 100 can be expected. Also particularly advantageous is the determination of a processing value 170 from the pressure signals 145 and 155 for the use of altitude information from the (ambient) pressure sensor, possibly in combination with an activity and location determination of the patient 100, to create a movement and fitness profile of the patient 100 to improve the control of the cardiac support system 105, but also to increase the safety of the patient 100.

With the approach presented here, it is thus also possible to determine blood pressure in patients with a non-pulsatile cardiac support system 105. A normal blood pressure measurement using an upper arm cuff does not provide any information about the blood pressure. By using multiple pressure sensors, for example, the sensors 135 in the ventricle 120 or 145 in the region of the aorta 130 (e.g., along a driveline), the velocity of the pulsatile components of the blood pumped by the heart 125 can be determined and incorporated as a factor into the assessment of the condition of the patient 100. The propagation speed of changes in the pump power of the cardiac support system 105 can be taken into account here as well.

The approach presented here also makes it possible to determine the differential pressure between the ventricle 120 and the aorta 130. As a result, a better adjusted pump output can be set, and the condition of the heart 125 and the cardiac support system 105 can be assessed, because the pressure output and the motor output of the motor 110, for example, can be correlated. The pulsatile changes or reactions to changes in the pump power imposed by the remaining power of the heart 125 can be used to infer the remaining elasticity of the blood vessels in the region of the cardiac support system 105.

The use of threshold values can furthermore also minimize side effects of the cardiac support system 105 (for example, keep ventricular and aortic blood pressure within the physiological range).

Additionally, in one design example of the approach presented here, long-term monitoring in an extracorporeal system or a device, such as the device 150 shown in FIG. 1 or a cloud, allows trend detection via the heart's 125 own capacity, and thus an assessment of the state of health of the patient 100. According to a further design example, long-term monitoring of the ventricular pressure data also enables an assessment of the condition of the heart 125; the force of contraction of the heart 125, for example, can be ascertained by evaluating δp/δt to determine the pressure increase at systole.

In a further design example, long-term monitoring of the end-diastolic pressure could also be used as a measure of the preload of the heart 125. Long-term monitoring of the pressure and motor data of the cardiac support system 105 can also be used as a measure of the remaining performance and lifespan of the cardiac support system 105.

According to a further design example, several or all values of the pressure sensors, i.e., of the pressure signals 145 or 155, can be transmitted from the intracorporeal control unit as the device 150 to an external device such as the cloud in order to prevent tampering with the pump or the cardiac support system 105 with respect to incorrect or abusive control of the pump output (in particular to ensure high IT security or to operate the cardiac support system 105 as an implant in read-only mode).

The cloud-based solution, especially in combination with a possible location determination of the patient 100, provides the option to increase blood flow, in particular for physically weaker patients 100, prior to strain, so that, for example, stairs can be negotiated more easily. In particular the altitude information that can be obtained from one of the pressure sensors 135 or 140 enables quick adjustment of support even without a cloud connection.

If many or all of the affected patients 100 are connected to the cloud, the algorithm can potentially predict dangerous strains on the hearts 125 of the patients and warn the patient 100, for example by learning specific patterns of the overall system, consisting of the physiological heart muscle 125 and the cardiac support system 105, and identifying said patterns early.

With real-time transmission of all data and immediate evaluation, the algorithm in the cloud can identify a potential failure of the cardiovascular system of the patient 100. In combination with the location determination, an ambulance can then be sent to the patient 100 in a fully automated manner. With this system, precious minutes can be gained in which rescue personnel are already on the way before a person accompanying the patient 100 makes the emergency call, or even before the person or patient 100 himself feels the life-threatening effects.

The ambient air pressure is still required, for example, to be able to determine the absolute blood pressure. FIG. 1 shows a simplified illustration of an implanted cardiac support system 105. The system 105 of FIG. 1 is located in the heart 125. It can also be connected via a lead to an intracorporeal control device or a device corresponding to the device 150 of FIG. 1. In this case, the pressure sensor for the ambient pressure is disposed in an extracorporeal component as the device 150, as shown in FIG. 1. In principle, the sensor signal could be transmitted as the first pressure signal 145 from the outside to the inside, i.e., from the device 150 shown in FIG. 1 to a device integrated into the cardiac support system 105, which is not explicitly shown in FIG. 1 for reasons of clarity. However, such a communication variant could allow tampering with the internal system, i.e., the cardiac support system 105. If the cardiac support system 105 is designed exclusively for communication from the inside out, tampering could be ruled out.

By combining cloud evaluation, location determination and blood pressure data, altitude profiles, walking speed and duration of the strain on the patient 100 can be synchronized very well with the respective occurring blood pressure values. Such an evaluation can help a physician to assess the state of health of the patient 100.

In one possible design example, the blood pressure data is acquired at a sampling rate in the range of 0.1 samples per second to 1000 samples per second, preferably with a sampling rate of 100 samples per second. In a further possible design example, the entire measurement data stream is transmitted from the intracorporeal system to the extracorporeal system. The measurement data can be compressed there, for example by lossy or loss-free compression.

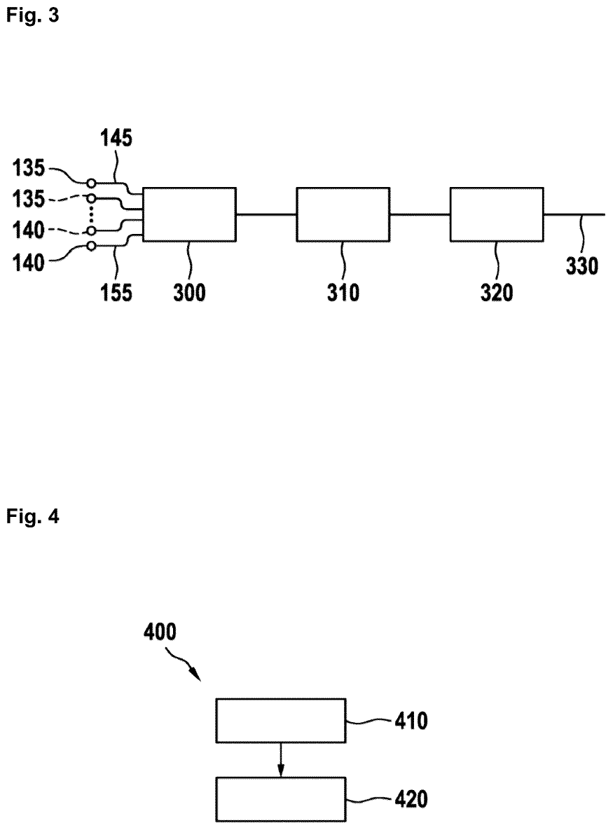

FIG. 3 shows a block diagram of an example of a compression method for streaming data, for example a blood pressure curve, to a cloud. Sensors (such as the pressure sensors 135 and 140 shown in FIG. 3) which are designed redundantly for reasons of reliability, for example, have obvious redundancy in the data signal, but the ventricular, aortic and ambient air pressure correlate as well. In order to minimize the bandwidth required to transmit data to the cloud, the data and/or the pressure signals 145 or 155 of the pressure sensors 135 or 140 can first be decorrelated in a decorrelation unit 300 and/or passed through a model-based predictor 310. A subsequent entropy coder 320 compresses the residual information of the blood pressure values and/or the pressure signals 145 and/or 155 before they are transmitted, for example as a compressed blood pressure curve 330, out of the patient 100 or into the cloud.

In addition to a model-based compression (with the predictor 320), as shown schematically in FIG. 3, a spectral compression, for example via a discrete cosine transformation or the use of wavelet filter banks, is possible as well.

Compression and transmission of the blood pressure curve using the pressure signals can take place in larger blocks of several seconds to minutes or continuously as a measurement data stream for instant display, for example on a physician's terminal. In one possible design example, the measurement data stream of (compressed and/or coded) pressure signals from the implanted sensors is already compressed in the implanted system or a cardiac support system 105 implanted in the patient 100 in order to reduce the required transmission bandwidth out of the body. In a further design example, several or all of the pressure sensors are designed redundantly. Redundant in this case means that at least two different or independent sensors can sense the same physiological variable. The sensor data of the redundant sensors can be transmitted as a whole. It is also possible to check whether the two sensor values are sufficiently identical (deviation below a threshold δε) in close proximity to the sensor. If the deviation is |p₁-p₂|<δϵ, only one sensor value is transmitted, for example an average value formed from the two values. If the deviation is outside the δϵ-range, an error code is transmitted. In the event of a fault, pressure signals can optionally still be transmitted in addition to the error code, for example the more plausible of the two pressures or the two pressures separately.

In a further design example, characteristic variables, for example diastolic, systolic and mean blood pressure, are already extracted from the blood pressure curve in the implanted cardiac support system 105 and transmitted to the extracorporeal system. The compensation of the air pressure and the further transmission or storage of the data takes place there. If there is strong variance in the blood pressure (e.g., due to large fluctuations in the air pressure resulting from technical systems such as defective air conditioners), the blood pressure data can be marked as invalid. For this purpose, the extracorporeal system identifies the fluctuation of the air pressure and compares it to a threshold value.

The selection of the operating mode (transmission of the blood pressure curve or the extracted characteristic parameters) can be based on fixed time intervals. For example, the averaged characteristic values are transmitted over a 5-minute interval and a detailed temporal progression of the blood pressure (blood pressure curve) is transmitted every 30 minutes for one minute. The selection of the operating mode can be triggered by the remote system (cloud, physician). Therefore, if the extracted characteristic parameters show abnormalities, the blood pressure curve can be obtained to carry out further diagnostics. Abnormalities could, for example, be a lack of sufficient pulsatility (difference between the diastolic and the systolic blood pressure), which could indicate a ventricular fibrillation, for example.

The measurement data can be obtained via a wireless modem (for example LoRa, NB-IoT, LTE, UMTS, GPRS) integrated into the device 150 or the cardiac support system. The use of the data connection of a portable mobile device (for example via Bluetooth, Wi-Fi or NFC) is possible as well. Routinely collected data can also initially be stored in the system (preferably the extracorporeal system) (in accordance with the methodology of a long-term ECG/Holter ECG). The stored data can be transmitted when a specific wireless network is available, at a specific time (e.g., at home at night) or in the physician's office. Here, too, the more current-intensive WAN wireless modem can be activated for immediate external assistance if an urgent need for action has been identified.

FIG. 4 shows a flow diagram of a design example of the approach presented here as the method 400 for monitoring a state of health of the patient. The method 400 comprises a step 410 of inputting a first pressure signal and a second pressure signal and a step 420 of processing the first pressure signal and the second pressure signal to determine a processing value in order to monitor the state of health of the patient based on said processing value.

If a design example includes an “and/or” conjunction between a first feature and a second feature, this should be read to mean that the design example according to one embodiment comprises both the first feature and the second feature and, according to another embodiment, comprises either only the first feature or only the second feature.

What is claimed is:

1. A cardiac support system comprising:
an intravascular blood guiding device comprising a blood pressure sensor configured to output a first pressure signal;
an ambient air pressure sensor configured to output a second pressure signal; and
a monitoring device configured to receiving and process the first pressure signal and the second pressure signal; and
wherein:
the intravascular blood guiding device is configured to be positioned within a heart; and
the monitoring device determines a change in a pressure within the heart based at least in part on the first pressure signal and the second pressure signal.

2. The cardiac support system of claim 1, wherein the intravascular blood guiding device is configured to be positioned across the aortic valve such that a first end is positioned within the ventricle and a second end is positioned within the aorta.

3. The cardiac support system of claim 2, wherein the intravascular blood guiding device is a blood pump comprising an inlet positioned near the first end and an outlet positioned near the second end, the blood pump configured to draw blood from the ventricle into the aorta.

4. The cardiac support system of claim 2, wherein the blood pressure sensor is disposed at the first end of the intravascular blood guiding device.

5. The cardiac support system of claim 2, wherein the blood pressure sensor is disposed at the second end of the intravascular blood guiding device.

6. The cardiac support system of claim 1, wherein the blood pressure sensor is a first blood pressure sensor, the intravascular blood guiding device further comprising a second blood pressure sensor configured to output a third pressure signal.

7. The cardiac support system of claim 5, wherein the monitoring device is configured to receive and process the third pressure signal and to determine a change in differential pressure between the ventricle and the aorta of the heart based at least in part on the first pressure signal, the second pressure signal, and the third pressure signal.

8. The cardiac support system of claim 1, wherein the ambient air pressure sensor is disposed within the monitoring device.

9. The cardiac support system of claim 1, wherein the monitoring device comprises an input interface for receiving the first pressure signal and the second pressure signal and a processing unit for processing the first pressure signal and the second pressure signal.

10. The cardiac support system of claim 1, wherein the monitoring device is a remote computer unit that can be contacted via an Internet connection.

11. A cardiac support system comprising:
a first blood pressure sensor disposed at a first end of an intravascular blood guiding device, the first blood pressure sensor configured to output a first pressure signal;
a second blood pressure sensor disposed at a second end of the intravascular blood guiding device, the second blood pressure sensor configured to output a second pressure signal;
an ambient air pressure sensor configured to output a third pressure signal; and
a monitoring device for receiving the first pressure signal, the second pressure signal, and the third pressure signal;
wherein the first pressure signal, the second pressure signal, and the third pressure signal are processed to determine a change in a differential pressure between a ventricle and an aorta of a heart based at least in part on the first pressure signal, the second pressure signal, and the third pressure signal.

12. The cardiac support system of claim 9, wherein the first pressure signal is indicative of a blood pressure within a ventricle, the second pressure signal is indicative of a blood pressure within an aorta, and the third pressure signal is indicative of an ambient air pressure.

13. The cardiac support system of claim 9, wherein the first end of the intravascular blood guiding device is configured to be positioned within the ventricle and the second end of the intravascular blood guiding device is configured to be positioned within the aorta.

14. The cardiac support system of claim 9, wherein the ambient air pressure sensor is disposed within the monitoring device.

15. The cardiac support system of claim 9, wherein the monitoring device comprises:
an input interface for receiving the first pressure signal, the second pressure signal, and the third pressure signal; and
a processing unit for processing the first pressure signal and the second pressure signal.

16. A method for monitoring a patient using a cardiac support system, the method comprising:
inserting the cardiac support system within a heart of the patient across the aortic valve, wherein the cardiac support system comprises a blood pressure sensor and a hose having an inlet and an outlet located proximal to the inlet;
providing an ambient air pressure sensor external to the patient;
receiving a first pressure signal from the blood pressure sensor and a second pressure signal from the ambient air pressure sensor; and
determining a change in pressure within the heart based at least in part on the first pressure signal and the second pressure signal.

17. The method of claim 15, wherein the blood pressure sensor is a first blood pressure sensor, wherein the cardiac support system further comprises a second blood pressure sensor.

18. The method of claim 16, further comprising receiving a third pressure signal from the second blood pressure sensor and determining a change in differential pressure between a ventricle and an aorta of the heart based at least in part on the first pressure signal, the second pressure signal, and the third pressure signal.

19. The method of claim 15, further comprising flowing blood from a ventricle to an aorta of the heart.

20. The method of claim 18, further comprising detecting a blood flow through the cardiac support system and receiving a blood flow signal indicative of the blood flow.
